(19) United States
(12) Patent Application Publication
(10) Pub. No.: US 2011/0207760 A1
(43) Pub. Date: Aug. 25, 2011

(54) SN-38 COMPOSITIONS

(76) Inventors: Valery Alakhov, Ile Bizard (CA); Grzegorz Pietrzynski, Montreal (CA); Patel Kishore, Pierrefonds (CA); Thomasz Popek, Pointe-Claire (CA)

(21) Appl. No.: 13/032,177

(22) Filed: Feb. 22, 2011

Related U.S. Application Data
(60) Provisional application No. 61/338,709, filed on Feb. 23, 2010.

(51) Int. Cl. A61K 31/4745; A61P 35/00

(52) U.S. Cl. 514/280

(57) ABSTRACT

In one aspect, the present invention is directed to a stable aqueous composition including (a) SN-38; (b) a sulfoalkyl derivative of a cyclodextrin; and (c) water; in which the concentration of SN-38 in the composition is at least about 0.13 mg/g of the composition. In another aspect, this invention is directed to a process for making the composition which process comprises mixing SN-38 and a sulfoalkyl derivative of a cyclodextrin in an aqueous medium wherein the water content of the mixture is less than about 60% by weight.

### CROSS REFERENCE TO RELATED APPLICATION

This application claims the benefit of U.S. Provisional Patent Application No. 61/338,709, filed Feb. 23, 2010, the entirety of which is hereby incorporated by reference into this application.

### FIELD OF THE INVENTION

In one aspect, the present invention is directed to a stable aqueous composition comprising (a) SN-38; (b) a sulfoalkyl derivative of a cyclodextrin; and (c) water; wherein the concentration of SN-38 in the composition is at least about 0.13 mg/g of the composition. In another aspect, this invention is directed to a process for making a composition which process comprises mixing SN-38 and a sulfoalkyl derivative of a cyclodextrin in an aqueous medium wherein the water content of the mixture is less than about 60% by weight.

### BACKGROUND OF THE INVENTION

SN-38, (4S)-4,11-Diethyl-4,9-dihydroxy-1H-pyrano[3′,4′:6,7]indolizino[1,2-b]quinoline-3,14(4H,12H)dione, also called 7-Ethyl-10-hydroxy-camptothecin, is known to exhibit anticancer activity. However, due to its extremely low solubility in aqueous solutions (SN-38 has a solubility of about 0.00008 mg/mL in water at room temperature), it is not feasible to provide SN-38 directly to cancer patients, as such low solubility prohibits the application of sufficient dose of SN-38 directly to the subject.

In therapy, SN-38 is used as a prodrug, Irinotecan, (S)-4,11-diethyl-3,4,12,14-tetrahydro-4-hydroxy-3,14-dioxo1H-pyrano [3′,4′:6,7]-indo lizino [1,2-b]quino lin-9-yl-[1,4′bipiperidine]-1′-carboxylate. Irinotecan is converted into SN-38 in the patient's body by the action of certain liver carboxylesterases. SN-38, which is believed to work by causing cell death via the inhibition of the topoisomerase I enzyme, is estimated to be up to 1000 times more active than is irinotecan itself. However, it is believed that the conversion of irinotecan to SN-38 is, in general, variable and inefficient, with only about 2% to about 8% of the prodrug being converted into the more active SN-38 molecule (Meyer-Losic, Clin. Cancer Res. 2008:14(7) pp. 2145-2153).

Several approaches to overcome the issues associated with irinotecan and/or the poor solubility of SN-38 have been proposed, including the use of alternative prodrugs (i.e., Meyer-Losic cited above); formulation with organic solvents (i.e., U.S. Pat. Nos. 5,447,936, 5,859,023, 5958,937, and 5,674,874); incorporation into nanoparticles (U.S. Patent Application Publication No. 2010/0008998) and incorporation into liposomes (U.S. Patent Application Publication No. 2003/0215492).

U.S. Pat. No. 6,653,319 (“the '319 patent”) discloses a process for the production of formulations of poorly water soluble camptothecin analogs (particularly DB-67) employing pH alteration in the presence of a solubilising agent such as a substituted cyclodextrin or a liposome. Exemplifying DB-67 and about 20% (w/v) of β-cyclodextrin derivatives, the '319 patent theorizes that concentrations up to 40% (w/v) of a β-cyclodextrin derivative can be prepared without drug precipitation. However, the sequential base/acid addition required by such method can result in the formation of undesirable amounts of salt, a result which may not be desired in a pharmaceutical product.

Accordingly, there is a need for formulations of SN-38 which can provide stable, efficient and predictable delivery of such compound for anticancer use.

### SUMMARY OF THE INVENTION

In one aspect, the present invention is directed to a stable aqueous composition comprising (a) SN-38; (b) a sulfoalkyl derivative of a cyclodextrin; and (c) water; wherein the concentration of SN-38 in the composition is at least about 0.13 mg/g of the composition.

In another aspect, the present invention is directed to a process for making a composition which process comprises mixing SN-38 and a sulfoalkyl derivative of a cyclodextrin in an aqueous medium wherein the water content of the mixture is less than about 60% by weight.

### DETAILED DESCRIPTION

In one aspect, the present invention is directed to a stable aqueous composition comprising (a) SN-38; (b) a sulfoalkyl derivative of a cyclodextrin; and (c) water; wherein the concentration of SN-38 in the composition is at least about 0.13 mg/g of the composition.

In another aspect, the present invention is directed to a process for making the composition which process comprises mixing SN-38 and a sulfoalkyl derivative of a cyclodextrin in an aqueous medium wherein the water content of the mixture is less than about 60% by weight.

The compositions of the present invention exhibit desirable stability in that they can be stored at room temperature for extended periods of time (of a day, a week or a month or more) without precipitation of the SN-38. By avoiding processes which require the use of acids and bases, the compositions of the present invention do not contain unnecessary amounts of salts which may be undesirable in a pharmaceutical product. Further the compositions of the present invention typically do not contain any organic solvent, although small amounts can be present so long as they do not affect the stability of the composition.

### SN-38

SN-38 or 7-Ethyl-10-hydroxy-camptothecin, is (4S)-4,11-Diethyl-4,9-dihydroxy-1H-pyrano[3′,4′:6,7]indolizino[1,2-b]quinoline-3,14(4H,12H)dione.

### Sulfoalkyl Derivatives of Cyclodextrins

Cyclodextrin is a cyclic oligo-1-4-alpha-D-glucopiranose comprising at least 6 sugar units. The most widely known are cyclodextrins containing six, seven or eight sugar units. Cyclodextrins containing six sugar units are known as alpha-cyclodextrins, those containing seven sugar units are known as beta-cyclodextrins and those consisting of eight sugar units are known as gamma-cyclodextrins. It is preferred that the cyclodextrins employed in the practice of this invention are beta-cyclodextrins.

The cyclodextrins which can be employed are modified with one or more sulfoalkyl groups attached to the sugar units by an ether bond substituting some of the hydroxyl groups of the cyclodextrin. Preferably, the number of modified sulfoalkyl groups per cyclodextrin molecule at least about 4; it is more preferred that the number of modified sulfoalkyl groups per cyclodextrin molecule is between about 6 and about 9; and it is particularly preferred that the average substitution is about 7. The sulfoalkyl cyclodextrins can be further modified by other chemical groups, such as for example hydroxyalkyl or poly(oxyethylene).

The sulfoalkyl groups of the cyclodextrins useful in the practice of this invention preferably comprise between 1 and 6 carbon atoms. It is more preferred that sulfoalkyl groups of the cyclodextrins employed in this invention are sulfobutyl.

Typically, the modified cyclodextrins are employed in the form of a salt. It is particularly preferred that the modified cyclodextrin employed in the invention is the sodium salt of sulfobutyl ether beta-cyclodextrin (SBECD).

### Preparation of the Composition of the Invention

The compositions of this invention can be prepared by mixing SN-38 and a sulfoalkyl derivative of a cyclodextrin in an aqueous medium wherein the water content of the mixture is less than about 60% by weight. Typically, the aqueous medium is deionized water. It is preferred that the SN-38 is pre-prepared as a fine powder, for example by grinding or micronization.

Preferably, the proportion of SN-38 to cyclodextrin, by weight, is between about 1:12,500 and about 1:25; is more preferably between about 1:5,000 and about 1:50; is even more preferably between about 1:2,500 and about 1:75 and most preferably between about 1:1,500 and about 1:100.

The water content of the mixture is less than about 60% by weight; is preferably less than about 50% by weight; and is most preferably about 40% by weight. The mixture must comprise sufficient amount of water to make the composition a liquid.

The mixture is then agitated for a sufficient period of time to ensure adequate mixing of the components. The amount of time required will depend upon the particular reaction conditions employed, and can range from less than a minute to several days or more. The extent of such mixing can be monitored by filtering a sample of the liquid and subjecting the sample to quantitative analysis, such as, for example HPLC. Ultrasound, homogenization, micronization, or other similar means can be employed to more rapidly mix the ingredients.

Although mixing can take place at room or slightly elevated temperatures, it is preferred that the mixture be heated to between about 80° and about 160° C.

Thus, in one preferred embodiment of this invention, the cyclodextrin/SN-38 mixture is placed in a sealed vessel to withstand increased pressure, or a vessel equipped with a condenser to prevent water evaporation. The vessel is heated (e.g., by heat exchange with heated medium) to between about 80° C. and about 160° C., preferably between about 100° C. and about 140° C., and is maintained at such for at least 1 minute.

In another preferred embodiment the mixture of aqueous SBECD and solid SN-38 is moved through heated pipes using pumps.

If subjected to heating, the product mixture is typically cooled down, and then filtered. Optionally a multi-step filtration is employed.

The process of the present invention provides a viscous liquid solution comprising SN-38 dissolved in concentrated aqueous solution of modified cyclodextrin. The solution is stable at room temperature and is suitable for a prolonged storage. The composition has a SN-38 concentration of at least about 0.13 mg/g or solution; preferably of at least about 0.15 mg/g of solution; more preferably of at least about 0.20 mg/g of solution; and even more preferably of at least about 0.40 mg/g of solution.

Optionally the solution can be used in further processes to obtain derivative products. The processes include, without limitation: dilution to obtain a less viscous liquid suitable for systemic dosing to patients; drying to obtain solid product; and mixing with other components to obtain a more complex mixture.

The invention can be further illustrated by the following examples thereof, although it will be understood that these examples are included merely for purposes of illustration and are not intended to limit the scope of the invention unless otherwise specifically indicated. All percentages, ratios, and parts herein, in the Specification, Examples, and Claims, are by weight and are approximations unless otherwise stated.

### EXAMPLES

### Example 1

Preparation of an SN-38 Composition in Aqueous 60% sulfobutyl ether beta-cyclodextrin (SBECD)

37.6 mL water was added to 62.4 g sodium sulfobutyl ether beta-cyclodextrin (having a substitution level of about 7, as determined by NMR, and a water content of 3.9%, as determined by Karl-Fisher method), and was mixed until homogenous. The resulting viscous liquid was filtered through a 0.2 micrometer nylon filter to produce 60% (w/w) clear aqueous solution of SBECD. The density of this solution was 1.29 g/mL.

2 mg of SN-38 was added to 4g of such 60% (w/w) aqueous SBECD, and the mixture heated in a boiling water bath for 1 hour, allowed to cool and kept at room temperature for 24 hours, and then filtered through a 0.2 micrometer nylon filter with a 1 micrometer glass fiber prefilter. A clear yellow viscous liquid was produced.

### Analysis

Aliquots of the resultant SN-38 composition in aqueous SBECD were diluted 6 times with water, and further diluted with 10% (w/w) aqueous SBECD to obtain samples having a concentration suitable for HPLC analysis. The samples were analyzed by HPLC using column Symmetry Shield C18 3.5 μ, 50×4.6 mm (Waters), water (0.1% TFA)—acetonitrile (0.1% TFA) gradient and UV detection at 225 nm and 260 nm. The concentrations of SN-38 in the samples were determined by comparison of the area under the peak with the results obtained from analysis of standard solutions.

The concentration of SN-38 in the product solution was 0.40 mg/g of composition.

### Example 2

Dissolution of SN-38 in 60% (w/w) Aqueous SBECD at 34° C.

A sample of 4 g of 60% (w/w) aqueous SBECD was mixed with 2 mg of SN-38, and the mixture was incubated at 34° C. with constant mixing for 48 hours. The mixture was then filtered through 0.2 micrometer nylon filter and analyzed by HPLC as described in Example 1. The concentration of SN-38 in solution was 0.16 mg/g.

### Example 3

### Dissolution of SN-38 in Aqueous SBECD at 100° C.

4 g samples of aqueous SBECD having a concentration of 20%, 30%, 40%, 50% and 60% (w/w), respectively, were mixed with 2 mg of SN-38. The mixtures were heated in boiling water bath for 1 hour and then were kept for 24 hours at room temperature. The mixtures were then filtered through a 0.2 micrometer nylon filter and analyzed by HPLC as described in Example 1. The concentration of SN-38 in tested solutions is presented in the Table 1 below:

TABLE 1

| SBECD | SN-38 concentration |
|---|---|
| concentration | [mg/g of solution] |
| 20% | 0.03 |
| 30% | 0.07 |
| 40% | 0.13 |
| 50% | 0.23 |
| 60% | 0.40 |

These results demonstrate that concentrated aqueous compositions of SN-38 can be prepared employing SBECD concentrations of 40% or more.

### Example 4

Physical stability of SN-38 in Diluted Aqueous Solutions of SBECD

The SN-38 solution prepared as in the Example 1 was diluted with water, by weight, to obtain solutions containing respectively 5% SBECD, 10% SBECD, and 20% SBECD. All solutions were kept at room temperature and periodically analyzed by HPLC using the method described in the Example 1. The relative change of SN-38 concentration calculated from the change of the area under the peak of SN-38 is presented in Table 2 below.

A solution of SN-38 in hydroxypropyl beta-cyclodextrin (HPbCD) was prepared using the procedure described in the Example 1. This solution was subsequently diluted with water, by weight, to obtain HPbCD concentration 20%. The relative change of SN-38 concentration in this solution after 70 hours was determined as described above, and is noted in Table 3 below.

A solution of SN-38 in dimethyl beta-cyclodextrin (DMbCD) was prepared using the procedure described in the Example 1. This solution was subsequently diluted with water, by weight, to obtain DMbCD concentration 20%. The relative change of SN-38 concentration in this solution after 22.5 hours was determined as described above, and is noted in Table 4 below.

TABLE 2

| | 5% SBECD | 10% SBECD | 20% SBECD |
|---|---|---|---|
| Time [h] | Concentration of SN-38 relative to time 0 h | | |
| 0 | 100.0 | 100.0 | 100.0 |
| 5 | 96.6 | 97.9 | 99.5 |
| 23 | 91.4 | 94.9 | 97.6 |
| 47 | 85.2 | 90.8 | 96.3 |
| 71 | 79.2 | 87.7 | 95.1 |
| 98 | 77.9 | 85.5 | 93.2 |

TABLE 3

| Time [h] | 20% HPbCD |
|---|---|
| 0 | 100.0 |
| 70 | 63.0 |

TABLE 4

| Time [h] | 20% DMbCD |
|---|---|
| 0 | 100.0 |
| 22.5 | 42.5 |

The results presented above demonstrate that compositions comprising SBECD are unexpectedly more stable than are those comprising other cyclodextrin derivatives.

### Example 5

Pharmacokinetics of SN-38 and SN-38G (the major metabolite of SN-38)

This study demonstrates the pharmacokinetics of SN-38 and SN-38 glucuronide (SN-38G, the major metabolite of SN-38) upon dosing of an SN-38 composition with 40% SBECD intravenously to rats.

Female Sprague-Dawley rats, 4 animals per group received i.v. injections of SN-38 (doses 0.65 mg/kg and 2 mg/kg) in 40% SBECD. The SN-38 solution for the treatment was prepared by the dilution of concentrated composition of the Example 1 with water. The blood plasma samples were collected from each group of animals, extracted using liquid extraction with cold methanol/acetonitrile mixture (1:1 v/v), and analyzed using a HPLC method with fluorescent detection. The results, including levels of SN-38 and SN-38G in plasma and calculated values of area under the curve (AUC) for SN-38 and SN-38G are presented in Tables 5 and 6 below.

TABLE 5
Pharmacokinetics of SN38 and SN-38G

Plasma levels [ng/mL]

| | | | |
|---|---|---|---|
| SN-38 | SN-38G | SN-38 | SN-38G |

| | | |
|---|---|---|
| | Dosing SN-38 | Dosing SN-38 |
| | 0.65 mg/kg | 2 mg/kg |
| Time [h] | in 40% SBECD | in 40% SBECD |

| | | | | |
|---|---|---|---|---|
| 0.17 | 157.2 | 266.5 | 625.0 | 1282.0 |
| 0.5 | 41.3 | 150.7 | 157.4 | 747.7 |
| 0.75 | 26.1 | 97.1 | 51.4 | 289.8 |
| 1 | 18.2 | 61.5 | 29.8 | 239.9 |
| 3 | 5.8 | 15.7 | 5.5 | 27.6 |
| 6 | 1.2 | 6.0 | 5.3 | 18.5 |

TABLE 6
AUC of SN-38

| | SN-38 | SN-38G | Ratio AUC |
|---|---|---|---|
| | AUC | AUC | SN-38/ |
| Dosing: | [ng*h/mL] | [ng*h/mL] | SN-38G |
| SN-38 0.65 mg/kg in 40% SBECD | 106 | 275 | 0.39 |
| SN-38 2 mg/kg in 40% SBECD | 323 | 1085 | 0.30 |

The above results demonstrate that the composition of the present invention provides the pharmaceutical agent (SN-38) with systemic bio availability as demonstrated by the formation of the main metabolite of the compound, SN-38G.

### Example 6

Effect of an SN38 composition on the Growth of Subcutaneous Solid Tumor of Human Colon Carcinoma LoVo DX Cells in Balb/c Mice

Lovo Dx cells (2.5×10 6 cells per an injection) in culture medium with 30% Matrigel were subcutaneously inoculated at 2 sides of the flank (in the mid-flank) of each of 15 Balb/c mice. 20 days after inoculation the animals were randomly divided into 2 groups: control (8 mice) and treated (7 animals). On day 21, 24, 27 and 30 after inoculation (days 1, 4, 7, and 10 of treatment), twice daily, at 10 am and at 4 pm, the animals received intraperitoneal injections. Control animals received each time injection of 0.49 mL of 0.9% saline. Treated animals received each time injection of SN-38 solution in 20% SBECD (w/w), 4.5 mg/kg of SN-38. The SN-38 solution for the treatment was prepared by the dilution of concentrated composition of the Example 1 with water. Tumor sizes and body weight of each animal was monitored during treatment. The results represented as average tumor volume estimated from measurements of tumor diameters (V=0.5*D1*D2*D2, where Dl and D2 are longer and shorter diameter of the tumor) are presented in Table 7 below (the standard error of mean is in parentheses).

TABLE 7
Tumor volume and its change upon treatment

| | Control group | | Treated group | |
|---|---|---|---|---|
| Day of | Tumor | Tumor | Tumor | Tumor |
| treat- | volume | volume | volume | volume |
| ment | [cm³] | increase* | [cm³] | increase* |
| 0 | 0.119 (0.009) | 100 | 0.123 (0.010) | 100 |
| 4 | 0.133 (0.013) | 110 (4) | 0.128 (0.010) | 105 (4) |
| 6 | 0.151 (0.013) | 128 (6) | 0.140 (0.011) | 115 (6) |
| 8 | 0.179 (0.016) | 151 (8) | 0.142 (0.012) | 117 (7) |
| 11 | 0.239 (0.021) | 202 (12) | 0.163 (0.017) | 133 (11) |
| 13 | 0.273 (0.026) | 233 (17) | 0.185 (0.019) | 150 (10) |
| 15 | 0.305 (0.030) | 261 (21) | 0.195 (0.020) | 157 (10) |
| 18 | 0.425 (0.051) | 364 (37) | 0.217 (0.024) | 175 (12) |
| 20 | 0.584 (0.063) | 504 (52) | 0.284 (0.040) | 224 (22) |
| 22 | 0.804 (0.091) | 686 (69) | 0.349 (0.055) | 271 (29) |
| p** | | | 0.024 | |

*Percentage relative to day 0 of treatment
**p - two-tailed paired t-test vs. control

This above results demonstrates that the composition of the present invention provides the pharmaceutical agent (SN-38) with systemic bioavailability as indicated by its anticancer activity.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A stable aqueous composition comprising:
(a) SN-38;
(b) a sulfoalkyl derivative of a cyclodextrin; and
(c) water;
wherein the concentration of SN-38 in the composition is at least about 0.13 mg/g of the composition.

2. The composition of claim 1 wherein component (b) is sulfobutyl ether beta-cyclodextrin.

3. The composition of claim 2 wherein the concentration of SN-38 in the composition is at least about 0.15 mg/g of the composition.

4. The composition of claim 3 wherein the concentration of SN-38 in the composition is at least about 0.20 mg/g of the composition.

5. The composition of claim 4 wherein the concentration of SN-38 in the composition is about 0.40 mg/g of the composition.

6. A process for producing an SN-38 formulation comprising mixing SN-38 and a sulfoalkyl derivative of a cyclodextrin in an aqueous medium wherein the water content of the mixture is less than about 60% by weight.

7. The process of claim 6 wherein the sulfoalkyl derivative of a cyclodextrin is sulfobutyl ether beta-cyclodextrin.

8. The process of claim 7 wherein the water content of the composition is less than about 50% by weight.

9. The process of claim 7 wherein the water content of the composition is about 40% by weight.

10. The process of claim 7 wherein the mixture is heated to between about 80° and about 160° C.

11. The process of claim 10 wherein the mixture is heated to between about 100° and about 140° C.
